(19) United States
(12) Patent Application Publication
(10) Pub. No.: US 2017/0156668 A1
(43) Pub. Date: Jun. 8, 2017

(54) SIMULTANEOUS BI-LATERAL BLOOD PRESSURE SYSTEM WITH POSITION INDICATOR

(71) Applicant: Abbas M. HUSAIN, Euclid, OH (US)

(72) Inventor: Abbas M. HUSAIN, Euclid, OH (US)

(21) Appl. No.: 14/957,404

(22) Filed: Dec. 2, 2015

(51) Int. Cl. A61B 5/00; A61B 5/022

(52) U.S. Cl. CPC A61B 5/684; A61B 5/02233; A61B 5/7425

(57) ABSTRACT

A simultaneous bi-lateral blood pressure system with position indicator has a dual input blood pressure monitor that allows a left and right blood pressure cuff to be plugged and to obtain left and right readings simultaneously. A position indicator attaches to a comfortable adjustable collar that attaches to a patient's neck and is retractably positioned to indicate the position of the reading to be taken. The blood pressure monitor displays systolic and diastolic readings as well as pulse. If there is a significant difference between the left and right readings, then further tests are ordered. The position indicator is marked with measurement lines to allow the user to record the position where the reading is being taken. This establishes a repeatable baseline where further readings are taken from the same position.

### BACKGROUND OF THE INVENTION

Blood pressure is an important part of a medical workup that provides medical professionals with information regarding the underlying health of a patient. Recent studies have suggested that taking the blood pressure simultaneously on both the left and right side of a patient can provide important information regarding heart disease or other cardiac problems. In an article published in THE LANCET, it was reported that differences between a patient's blood pressure from each arm could help identify patients who could be at risk for heart problems (The Lancet, Volume 379, Issue 9819, Pages 905-914, 10 Mar. 2012; also from the Harvard Medical School http://www.health.harvard.edu/blog/different-blood-pressure-in-right-and-left-arms-could-signal-trouble-201202014174). It is well known that when a patient has a great differential blood pressure bilaterally, it is likely a sign of some serious vascular problem such as aneurysm or constriction of aorta or other vascular problem. If a medical professional wants this information, they have to repeat the blood pressure test on each side which is time consuming and also can lead to inaccuracy because of the time differential between readings.

There is a need for a blood pressure system that allows the practitioner to compare the blood pressure readings from all four quadrants of the patient's body.

### SUMMARY OF THE INVENTION

A simultaneous bi-lateral blood pressure system with position indicator has a dual input blood pressure monitor that allows a left and right blood pressure cuff to be plugged and to obtain left and right readings simultaneously. A position indicator attaches to a comfortable adjustable collar that attaches to a patient's neck and is retractably positioned to indicate the position of the reading to be taken. The blood pressure monitor displays systolic and diastolic readings as well as pulse. If there is a significant difference between the left and right readings, then further tests are ordered. The position indicator is marked with measurement lines to allow the user to record the position where the reading is being taken. This establishes a repeatable baseline where further readings are taken from the same position.

Other features and advantages of the instant invention will become apparent from the following description of the invention which refers to the accompanying drawings.

### BRIEF DESCRIPTION OF THE DRAWINGS

FIG. 1 is an illustration of a simultaneous bi-lateral blood pressure system with position indicator according to an embodiment of the invention.

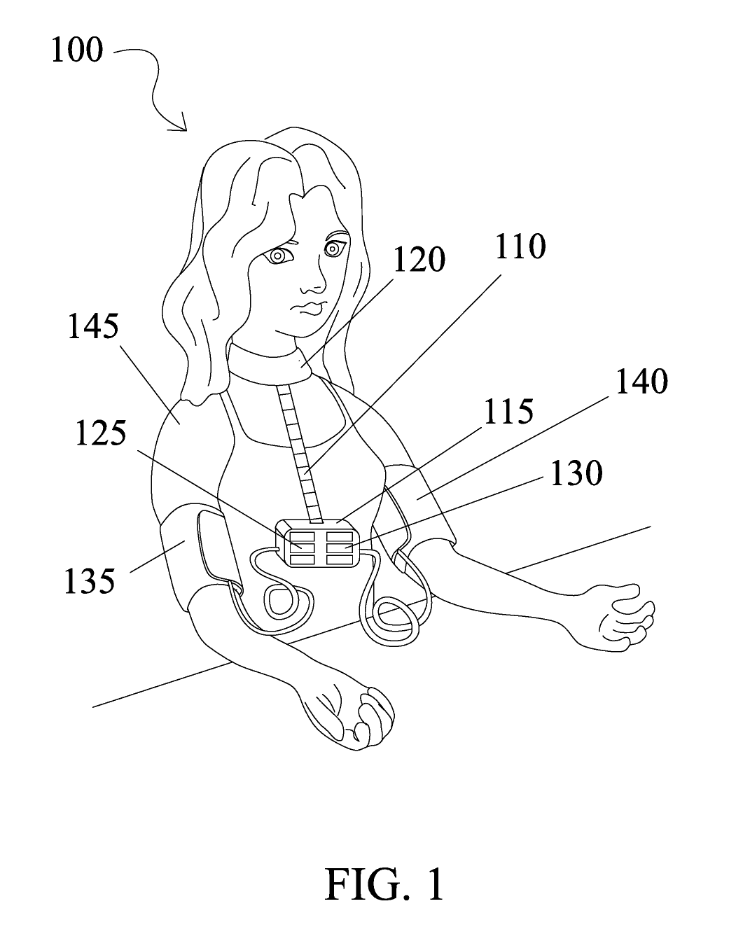

FIG. 2 is an illustration of the simultaneous bi-lateral blood pressure system with position indicator at an alternative blood pressure location.

FIG. 3 is an illustration of the simultaneous bi-lateral blood pressure system with position indicator at another alternative blood pressure location.

FIG. 4 is an illustration of the simultaneous bi-lateral blood pressure system with position indicator at yet another alternative blood pressure location.

### DETAILED DESCRIPTION OF THE INVENTION

In the following detailed description of the invention, reference is made to the drawings in which reference numerals refer to like elements, and which are intended to show by way of illustration specific embodiments in which the invention may be practiced. It is understood that other embodiments may be utilized and that structural changes may be made without departing from the scope and spirit of the invention.

Referring to the figures, a simultaneous bi-lateral blood pressure system with position indicator 100 is shown having a dual input blood pressure monitor 115 which has a position indicator 110 built in in order to allow a user to record the approximate location where the bi-lateral blood pressure measurements are being taken. This can provide a more detailed baseline as well as helping diagnosis other medical issues that might need to be explored if the readings suggest a problem. Position indicator 110 is flexible and rests against a patient 145 and is spring biased to retract and is contained within blood pressure monitor 115. Retracting systems are well known.

A measurement anchoring collar 120 is used to provide a solid pulling point for position indicator 110. Measurement anchoring collar is made of a soft material and is affixed using a hook and loop connector (not shown) to allow it to adjust to the patient's neck. Distance indicator lines are provided on position indicator 110 to allow the user to record the approximate position with respect to patient's body in order to provide consistent readings and to rule out other cause for differential readings. This is helpful where a user wants to track blood pressure over time by knowing where the readings are being taken from and to take all future readings from the same position. Of course other means may be used to allow position indicator 110 to function such as, but not limited to a pin, clip-on mount, lanyard, etc. as long as the end of position indicator 110 is held in place while moving blood pressure monitor 115 to the desired position for the readings to be taken. A single activator (not shown) is used to simultaneously energize both sides to provide simultaneous bi-lateral readings.

A left blood pressure cuff 140 is used to take the reading on the left side and a right cuff 135 is used for the right side. The cuffs (140 and 135) are attached using a hook and loop fastener and are connected to blood pressure monitor 115 with tubing as is known in the art.

Blood pressure monitor 115 has a left display 130 and a right display 125. Both displays (130 and 125) are used to display blood pressure information as well as pulse. Displays 130 and 125 display patient's vital signs. Both the diastolic and systolic pressures are indicated. The instant device allows the health professional to simultaneously take and compare the left side readings with the right side readings and if there is a significant differential, then further tests such as angiogram, CT scan, MRI or MRA or other tests as is known in the art. Individuals with diabetes, smokers or people with genetic predisposition for blood flow constriction are of particular concern. The instant invention can discover these issues before other symptoms become evident thus making the instant invention extremely valuable for quick, easy and inexpensive diagnostic procedures.

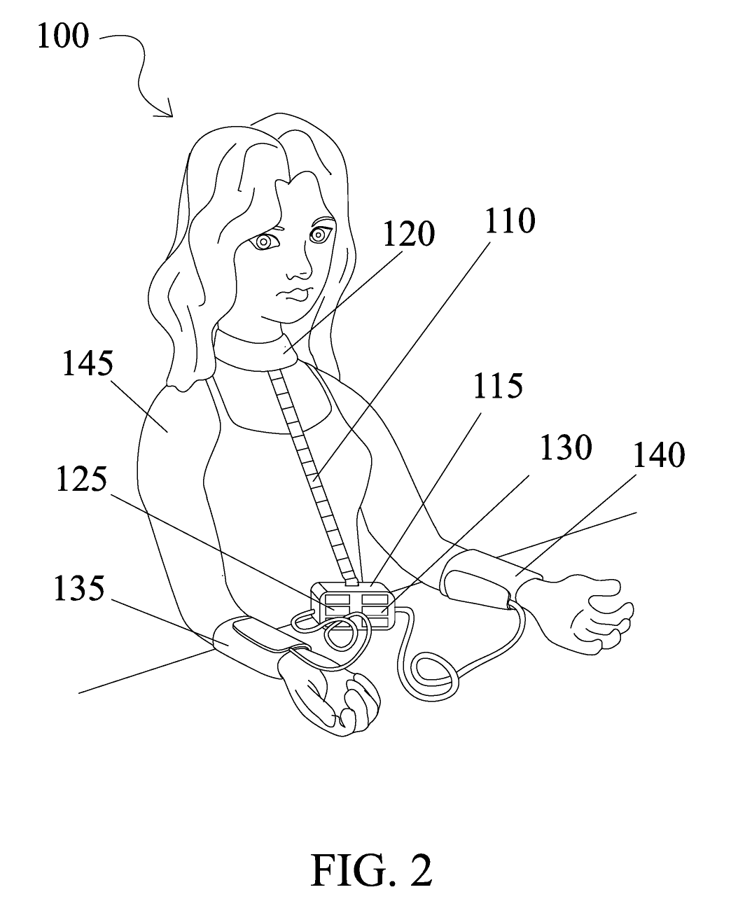

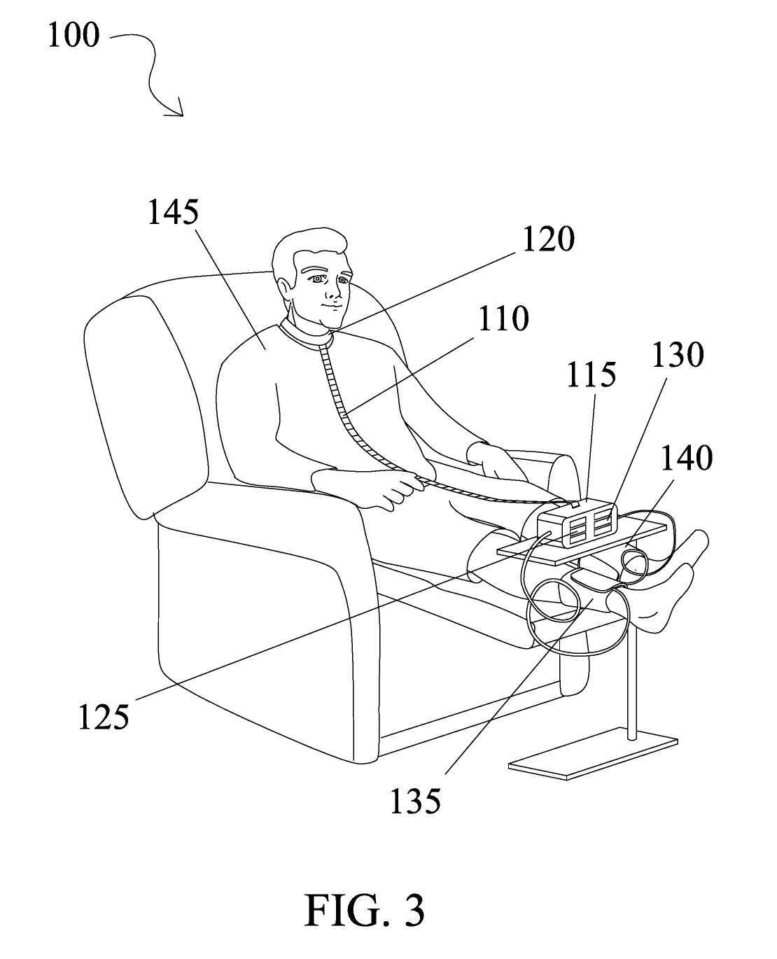

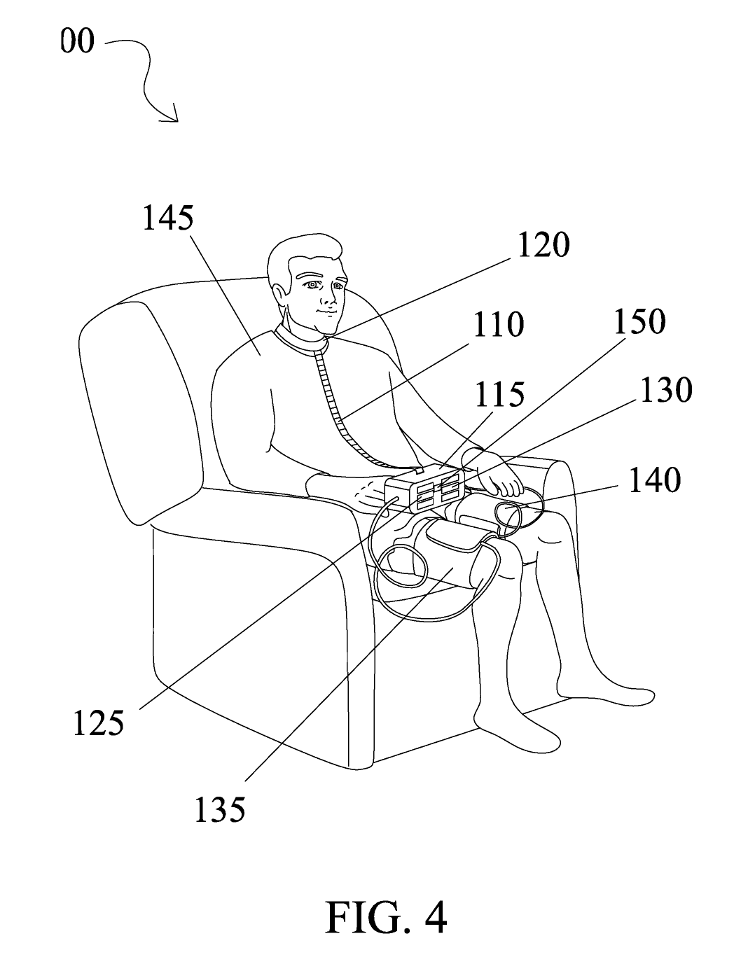

FIGS. 2, 3 and 4 shows blood pressure cuffs 140 and 135 located on the wrists rather than the upper arm. FIGS. 3 and 4 illustrate taking the readings on the lower extremities. Of course the position where the readings are taken can vary and the positions shown are merely for illustrative purposes only and not meant to limit the positions to only the 4 locations shown.

Additionally, although blood pressure monitor is shown having left and right displays (130 and 125) built in, it is known to wirelessly connect to a remote display and although not shown, any display function is acceptable as long as 2 simultaneous blood pressure readings are obtained. Also, although position indicator 110 is shown as being a tape measure, the position could be electronically achieved for example; by using an infra-red positioning device with a transmitter attached to the patient's neck and blood pressure monitor 115 receiving the signal where it is interpreted as distance. Additionally, a digital display 150 may be used to graphically display position or any other information that is desired. Dual input blood pressure monitor 115 may be battery operated or plug into an AC outlet.

Various pathologies such as constriction, dilation or obstruction occur in the middle of the aorta for the upper extremities or in the artery going down to the legs. For the upper extremities, there is a common arch of aorta radiating from the heart with one radial artery providing blood to the right arm and another radial artery for the left arm. If there is a significant differential between the two readings, then the user can order further tests such as angiogram, CT scan, MRI or MA for example to determine if there is truly a problem.

Although the instant invention has been described in relation to particular embodiments thereof, many other variations and modifications and other uses will become apparent to those skilled in the art.

What is claimed is:

1. A simultaneous bi-lateral blood pressure system with position indicator comprising:
a dual input blood pressure monitor;
a measurement anchoring collar;
a position indicator retractably disposed between said measurement anchoring collar and said blood pressure monitor;
a left blood pressure cuff adapted to connect to a left input on said dual input blood pressure monitor;
a right blood pressure cuff adapted to connect to a right input on said dual input blood pressure monitor;
a data display means for displaying and
said position indicator being adapted to display an approximate position of said dual input blood pressure monitor from a reference point wherein additional measurements are referenced and wherein a simultaneous measurement of both a left blood pressure reading and a right blood pressure reading are compared to provide diagnostic information.

2. The simultaneous bi-lateral blood pressure system with position indicator according to claim 1 wherein said dual input blood pressure monitor is adapted to display systolic blood pressure, diastolic blood pressure and pulse.

3. The simultaneous bi-lateral blood pressure system with position indicator according to claim a wherein said position indicator is a flexible ruler.

4. The simultaneous bi-lateral blood pressure system with position indicator according to claim 1 further comprising a digital readout to indicate said approximate position of said dual input blood pressure monitor.

5. The simultaneous bi-lateral blood pressure system with position indicator according to claim 1 wherein
